(19) United States
(12) Patent Application Publication
(10) Pub. No.: US 2011/0054630 A1
(43) Pub. Date: Mar. 3, 2011

(54) ARTIFICIAL BONE COATED WITH APATITE/COLLAGEN COMPOSITE, AND ITS PRODUCTION METHOD

(75) Inventor: Daisuke Shoji, Tokyo (JP)

(73) Assignee: HOYA CORPORATION, Tokyo (JP)

(21) Appl. No.: 12/990,566

(22) PCT Filed: Apr. 24, 2009
(86) PCT No.: PCT/JP2009/058145

§ 371 Date: Nov. 1, 2010

(30) Foreign Application Priority Data
May 7, 2008 (JP) 2008-121396

(51) Int. Cl. A61F 2/28; B05D 7/00

(52) U.S. Cl. 623/23.6; 427/2.26

(57) ABSTRACT

An artificial bone comprising a support coated with an apatite/collagen composite, and a method for producing the artificial bone by immersing the support in a dispersion of the fibrous apatite/collagen composite and drying it.

### FIELD OF THE INVENTION

The present invention relates to an artificial bone coated with an apatite/collagen composite.

### BACKGROUND OF THE INVENTION

Bone defect portions generated by bruise or illness are now treated by implanting patients' autogenous bones, similar bones provided by others, artificial bones made of metals such as titanium or hydroxyapatite ceramics, etc. Among them, hydroxyapatite ceramics having bone conduction not achieved by conventional metals, polymers or alumina ceramics and directly bonding to bones have been gradually finding wider use as bone-repairing materials substituting autogenous bones in various fields such as oral surgery, neurological surgery, otorhinolaryngology, plastic surgery, etc., since their commercialization. However, artificial bones made of ceramics such as hydroxyapatite are hard and brittle, disadvantageous in difficulty in handling during operation.

For easy handling, apatite/collagen composites having sponge-like elasticity have been developed. For example, as an artificial bone degradable in a living body having a closer composition to the autogenous bone than an artificial apatite bone, U.S. Pat. No. 5,776,193 A discloses a porous body having a network comprising hydroxyapatite to which collagen and if necessary other binders are bonded. Because of biodegradability, this porous body is absorbed by the body with autogenous bone formed therein. Accordingly, it can be used for fixing the spine, filling bone defects, curing bone fracture, implanting in periodontal osseous defects, etc. However, the apatite/collagen composite described in U.S. Pat. No. 5,776,193 A has such low strength that it cannot be used in portions subject to load.

On the other hand, metals, etc. used in portions subject to load have poorer bone conduction than that of artificial apatite ceramic bone, having poorer osseointegration.

### OBJECT OF THE INVENTION

Accordingly, an object of the present invention is to provide an artificial bone comprising a support coated with an apatite/collagen composite to have such strength as to be usable in portions subject to load, and having excellent bone conduction, and its production method.

### DISCLOSURE OF THE INVENTION

As a result of intensive research in view of the above object, the inventors have found that an artificial bone comprising a support made of a ceramic, a metal or a polymer coated with an apatite/collagen composite has excellent bone conduction. The present invention has been completed based on such finding.

Thus, the artificial bone of the present invention comprises a support coated with an apatite/collagen composite.

The support is preferably made of a ceramic, a metal or a polymer. The ceramic is preferably calcium phosphate.

The support is preferably a porous body, and the inner walls of pores in the porous body are preferably coated with the apatite/collagen composite.

The method of the present invention for producing an artificial bone comprising a support coated with an apatite/collagen composite comprises the steps of immersing the support in a dispersion of the fibrous apatite/collagen composite, and drying it.

The immersion is conducted preferably under reduced pressure.

### DESCRIPTION OF THE BEST MODE OF THE INVENTION

[1] Artificial Bone

The artificial bone of the present invention is obtained by coating a support with a self-organized apatite/collagen composite. Because the apatite/collagen composite is preferable as a biomaterial, the artificial bone of the present invention has excellent biocompatibility and bone conduction. By sterilization with γ-rays, electron beams, dry-heating, etc., it can be used as a bone-regenerating material, etc. in portions subject to load.

Though not restrictive, the support for forming the artificial bone may be made of a ceramic, a metal, a polymer, etc. The ceramic is preferably calcium phosphate. The metal is preferably titanium. In the case of using a polymer, the polymer per se preferably has osseointegration, particularly it is preferably polylactic acid. The support surface preferably has some roughness in order that it is easily coated with an apatite/collagen composite, providing high bone conduction.

The support is preferably a porous body. Though not particularly restricted, the porous body preferably has porosity of 30-92%. With the porosity in the above range, the artificial bone can bear a relatively large amount of an apatite/collagen composite in pores while keeping its mechanical strength, resulting in excellent bone conduction. The porosity is more preferably 50-90%. The porous body preferably has communicating pores. In the case of having communicating pores, the apatite/collagen composite can be present inside the porous body.

In the artificial bone, the apatite/collagen composite may densely fill pores, or may expand as thin films on the inner walls of pores. When the apatite/collagen composite expands as thin films on the inner walls of pores, the body fluid can flow easily, accelerating the formation of the bone. The support may have any shape necessary for treating the affected part of the body, for example, a cube, a prism, a cylinder, a screw, a pin, a washer, and granules.

[2] Production Method

The artificial bone of the present invention is obtained by coating a support with an apatite/collagen composite. The apatite/collagen composite is preferably a composite similar to the living bone, in which hydroxyapatite and collagen are oriented in a self-organized manner. The term “self-organized” used herein means that calcium hydroxyphosphate having an apatite structure (hydroxyapatite) has orientation along collagen fibers, peculiar to the living bone; the C-axis of hydroxyapatite being orientated along collagen fibers.

(1) Production Of Apatite/Collagen Composite

(a) Starting Materials

The apatite/collagen composite is produced from collagen, a phosphate and a calcium salt. The collagen may be extracted from animals, etc., though their kinds, parts, ages, etc. are not particularly restrictive. In general, collagen obtained from skins, bones, cartilages, tendons, internal organs, etc. of mammals such as cow, pig, horse, rabbit and rat and birds such as hen, etc. may be used. Collagen-like proteins obtained from skins, bones, cartilages, fins, scales, internal organs, etc. of fish such as cod, flounder, flatfish, salmon, trout, tuna, mackerel, red snapper, sardine, shark, etc. may also be used. The extraction method of collagen is not particularly restrictive but may be a usual one. In place of collagen extracted from animal tissues, collagen produced by gene recombination technologies may also be used.

The phosphoric acid or its salt [hereinafter simply called “phosphoric acid (salt)”] may be phosphoric acid, disodium hydrogenphosphate, sodium dihydrogenphosphate, dipotassium hydrogenphosphate, potassium dihydrogenphosphate, etc. The calcium salts may be calcium carbonate, calcium acetate, calcium hydroxide, etc. The phosphate and the calcium salt are preferably added in the form of a uniform aqueous solution or suspension.

From the aspect of mechanical strength, a mass ratio of apatite to collagen in the apatite/collagen composite used in the present invention is preferably 9/1 to 6/4, more preferably 8.5/1.5 to 7/3, most preferably about 8/2.

(b) Preparation Of Solution

An aqueous solution of collagen and phosphoric acid (salt) is generally prepared by adding an aqueous collagen solution to an aqueous phosphoric acid (salt) solution. The concentration of collagen in the aqueous solution of collagen and phosphoric acid (salt) is preferably 0.1-1.5% by mass, particularly about 0.85% by mass. The concentration of phosphoric acid (salt) is preferably 15-240 mM, particularly about 120 mM. The aqueous collagen solution used preferably contains about 0.85% by mass of collagen and about 20 mM of phosphoric acid. The concentration of an aqueous calcium salt solution (or suspension) is preferably 50-800 mM, particularly about 400 mM. The fiber length of the apatite/collagen composite can be controlled by adjusting the concentration of each solution. Specifically, a higher concentration of each solution provides shorter fibers, while a lower concentration of each solution provides longer fibers

(c) Synthesis Method

An aqueous solution of collagen and phosphoric acid (salt), and an aqueous calcium salt solution or suspension are simultaneously dropped into water substantially in the same amount as that of the aqueous calcium salt solution or suspension added, at about 40° C. to form an apatite/collagen composite. The fiber length of the apatite/collagen composite can be controlled by adjusting dropping conditions. The dropping speed is preferably 1 to 60 mL/minute, more preferably about 30 mL/minute. The stirring speed is preferably 1 to 400 rpm, more preferably about 200 rpm. The mixing ratio of the phosphoric acid (salt) to the calcium salt is preferably 1/1 to 2/5, more preferably 3/5. The mixing ratio of collagen to apatite (the total of the phosphoric acid salt and the calcium salt) is preferably 1/9 to 4/6, more preferably 1.5/8.5 to 3/7.

The reaction solution is preferably kept at pH of 8.9 to 9.1 by maintaining a calcium ion concentration at 3.75 mM or less and a phosphoric acid ion concentration at 2.25 mM or less. Outside the above concentration ranges of the calcium ion and/or the phosphoric acid ion, the self-organization of the composite would be hindered. The above dropping conditions provide the self-organized apatite/collagen composite with fiber length of 2 mm or less suitable as a powdery apatite/collagen material.

After the completion of dropping, a slurry-like, aqueous apatite/collagen composite dispersion is freeze-dried. The freeze-drying is carried out by rapid drying in vacuum in a frozen state at −10° C. or lower.

(2) Production Of Artificial Bone

(a) Preparation Of Apatite/Collagen Composite Dispersion

The apatite/collagen composite powder is mixed with water, a physiological saline solution, etc., and stirred to prepare a dispersion. The concentration of the apatite/collagen composite in the dispersion may be properly determined depending on how much composite the support is coated with, but it is preferably 1-30% by mass, more preferably 3-10% by mass. The amount of the dispersion applied to the support can be controlled by adjusting the viscosity of the dispersion. When the dispersion is stirred after adding a liquid, part of apatite/collagen composite fibers are cut, providing a larger fiber length distribution. By adjusting the stirring condition, the resultant coating is provided with improved strength.

To improve the adhesion of the apatite/collagen composite to the support, a binder is preferably added to the dispersion. The binders may be soluble collagen, gelatin, polylactic acid, polyglycolic acid, copolymers of lactic acid and glycolic acid, polycaprolactone, carboxymethylcellulose, cellulose esters, dextrose, dextran, chitosan, hyaluronic acid, Ficoll, chondroitin sulfate, polyvinyl alcohol, polyacrylic acid, polyethylene glycol, polypropylene glycol, water-soluble polyacrylate, water-soluble polymethacrylate, etc., and the soluble collagen is particularly preferable. The amount of the binder added is preferably 0.1-10% by mass, more preferably 0.5-5% by mass, based on 100% by mass of the apatite/collagen composite.

As in the production of the apatite/collagen composite, the binder is added preferably in the form of an aqueous phosphoric acid solution. The concentration, etc. of the binder solution added are not particularly restrictive, but practically the concentration of the binder is preferably about 0.85% by mass, and the concentration of phosphoric acid is preferably about 20 mM.

After adding an aqueous solution of the binder in phosphoric acid (salt), the pH of the dispersion is adjusted to about 7 by an aqueous sodium hydroxide solution. To prevent collagen from becoming gelatin in the gelation described later, the pH of the dispersion is adjusted to preferably 6.8-7.6, more preferably 7.0-7.4.

To accelerate collagen added as the binder to become fibers, the dispersion is mixed with a concentrated (about 10 times) solution of a physiological buffer saline (PBS) of phosphoric acid to adjust the ionic strength of the dispersion to 0.2 to 1. The more preferred ionic strength is as large as about 0.8, on the same level as that of PBS.

Additives such as antibiotics (tetracycline, etc.), chemotherapeutic agents (cisplatin, etc.), bone marrow cells, cell-proliferating factors (BMP, FGF, TGF-β, IGF, PDGF, VEGF, etc.), physiological activation factors (hormone, cytokine, etc.), etc. may be added to the dispersion within a range not hindering the object of the present invention.

(b) Coating To Support

The support is immersed in the resultant dispersion to coat the support with the apatite/collagen composite. When the support is a porous body, the support is preferably subject to reduced pressure in a vacuum desiccator, etc. in a state that it is immersed in the dispersion, such that the dispersion penetrate into pores. The amount of the apatite/collagen composite applied can be controlled by adjusting the viscosity of the dispersion and the speed of pulling up the support from the dispersion. The dry thickness of the apatite/collagen composite is preferably 1-3000 μm, more preferably 300-1000 μm.

(c) Gelation Of Dispersion

The dispersion applied to the support is kept at 35-45° C. to turn collagen added as a binder to fibers, thereby gelating the dispersion. Gelation provides a uniform, porous coating. The heating temperature is more preferably 35-40° C., and the heating time is preferably 0.5-3.5 hours, more preferably 1-3 hours.

(d) Freeze Drying

The dispersion applied to the support is frozen. The freezing temperature is preferably −80° C. to −10° C., more preferably −80° C. to −20° C. The sizes and shapes of pores in the coating can be controlled by a freezing speed. For instance, a larger freezing speed tends to provide smaller pore sizes to the resultant porous body. The coating is freeze-dried by evacuating and rapidly drying in a frozen state at −10° C. or lower, as in the case of the composite. As long as the dispersion is sufficiently dried, the freeze-drying time is not particularly restricted, but generally about 24 to 72 hours.

(e) Cross-Linking Of Collagen

To provide the coating of the artificial bone with increased mechanical strength and shape retention for a desired period of time when implanted in a human body, it is preferable to cross-link collagen in the coating. The cross-linking of collagen may be carried out by any methods such as physical cross-linking methods using γ-rays, ultraviolet rays, electron beams, thermal dehydration, etc., or chemical cross-linking methods using cross-linking agents, condensation agents, etc. The chemical cross-linking can be conducted, for instance, by a method of immersing the freeze-dried coating in a cross-linking agent solution, a method of bringing the freeze-dried coating into contact with a steam containing a cross-linking agent, or a method of adding a cross-linking agent to an aqueous solution or suspension of the apatite/collagen composite in the course of its production process.

The cross-linking agents may be aldehydes such as glutaraldehyde, formaldehyde, etc.; isocyanates such as hexamethylene diisocyanate, etc.; carbodiimides such as a hydrochloric acid salt of 1-ethyl-3-(3-dimethylaminopropyl)carbodiimide; epoxies such as ethylene glycol diethyl ether, etc.; transglutaminase, etc. Among these cross-linking agents, glutaraldehyde is particularly preferable from the aspect of the easiness of controlling the degree of cross-linking and the compatibility of the cross-linked, apatite/collagen coating with a human body.

When the coating is immersed in a glutaraldehyde solution for cross-linking, the concentration of the glutaraldehyde solution is preferably 0.005 to 0.015% by mass, more preferably 0.005 to 0.01% by mass. When an alcohol such as ethanol is used as a solvent for glutaraldehyde, dehydration occurs simultaneously with the cross-linking of collagen. Accordingly, a cross-linking reaction occurs in a state where the apatite/collagen composite is contracted, resulting in a cross-linked, apatite/collagen coating with improved elasticity.

After the cross-linking, the apatite/collagen coating is immersed in an aqueous solution of about 2% by mass of glycine to remove unreacted glutaraldehyde, and then washed with water. It is further immersed in ethanol for dehydration, and then dried at room temperature.

The coated artificial bone may be sterilized with ultraviolet rays, γ-rays, electron beams, dry-heating, etc.

The present invention will be explained in more detail referring to Examples without intention of restricting it thereto.

### EXAMPLE 1

400 ml of a 120-mM aqueous phosphoric acid solution was added to 412 g of an aqueous collagen solution containing phosphoric acid (0.97% by weight of collagen, and 20 mM of phosphoric acid), and stirred to obtain a solution I. 400 ml of a 400-mM calcium hydroxide solution (solution II) was also prepared. Both solutions I and II were simultaneously dropped into a vessel containing 200 ml of water to obtain a dispersion of an apatite/collagen composite. Incidentally, the reaction solution was stirred at 200 rpm, and the dropping speed of the solutions I and II was adjusted to about 30 mL/min such that the reaction solution was kept at pH of 8.9-9.1. The fiber length of the resultant apatite/collagen composite was about 2 mm or less. The slurry was freeze-dried. The mass ratio of apatite to collagen in the composite was 8/2.

40 g of dried apatite/collagen composite fibers and 2 mL of a 1-N, pH-adjusting, aqueous sodium hydroxide solution, and 66 g of an aqueous collagen solution containing phosphoric acid (0.58% by mass of collagen, and 20 mM of phosphoric acid) for a binder were added to 500 ml of a physiological saline solution, and stirred at 10000 rpm for 3 minutes to prepare a dispersion of the apatite/collagen composite. A support formed by calcium phosphate having porosity of 85% was immersed in this dispersion, and subject to reduced pressure in a vacuum desiccator such that the dispersion fully penetrated into the porous body. After taken out of the dispersion, the apatite/collagen composite dispersion coated on the support was subject to gellation at 37° C. for 2 hours, cooled at −5° C. for 12 hours, and then frozen at −30° C. After drying the frozen coating by a freeze drier, it was subject to cross-linking by thermal dehydration in vacuum at 140° C. for 12 hours to obtain an artificial bone coated with the apatite/collagen composite. The artificial bone was broken at an arbitrary position to see a fractured surface by SEM. As a result, apatite/collagen composite coatings were confirmed in the pores.

### EFFECT OF THE INVENTION

The artificial bone of the present invention has excellent bone conduction, because it is coated with an apatite/collagen composite having bone-like structure and composition. The formation of the support by ceramics, metals or polymers enables it to be used in portions subject to load.

What is claimed is:

1. An artificial bone comprising a support coated with an apatite/collagen composite.

2. The artificial bone according to claim 1, wherein said support is made of a ceramic a metal or a polymer.

3. The artificial bone according to claim 2, wherein said ceramic is calcium phosphate.

4. The artificial bone according to claim 1, wherein said support is a porous body with inner walls of pores coated with the apatite/collagen composite.

5. A method for producing an artificial bone comprising a support coated with an apatite/collagen composite, comprising the steps of immersing the support in a dispersion of the fibrous apatite/collagen composite, and drying it.

6. The method according to claim 5, wherein said immersion is conducted under reduced pressure.
